(19) United States
(12) Patent Application Publication
(10) Pub. No.: US 2007/0166346 A1
(43) Pub. Date: Jul. 19, 2007

(54) Method for coating an object with hydrophobin at low temperatures

(75) Inventors: Rick Rink, Groningen (NL); Karin Scholtmeijer, Groningen (NL)

Correspondence Address: TRASK BRITT, P.O. BOX 2550, SALT LAKE CITY, UT, 84110, US

(73) Assignee: Applied NanoSystems B.V., Groningen (NL)

(21) Appl. No.: 11/485,186

(22) Filed: Jul. 12, 2006

Related U.S. Application Data
(63) Continuation of application No. PCT/NL05/00022, filed on Jan. 14, 2005.

(30) Foreign Application Priority Data
Jan. 16, 2004 (EP) 04075107.5

(51) Int. Cl. A61K 31/74; A61F 2/02

(52) U.S. Cl. 424/423; 424/78.09; 427/2.24

(57) ABSTRACT

The invention relates to methods for coating objects with hydrophobins. Provided is a method for providing the surface of an object with a hydrophobin coating, comprising contacting at least a part of an object with a hydrophobin-containing solution to form a hydrophobin layer on the surface of the object and exposing the layer to a pH below 7, preferably below 4, more preferably below 2, optionally in the presence of a detergent. Contacting can be performed at around room temperature and the hydrophobin-containing solution can be a supernatant of a culture medium of an organism that secretes a hydrophobin.

### CROSS-REFERENCE TO RELATED APPLICATIONS

This application is a continuation of PCT International Patent Application No. PCT/NL2005/000022, filed Jan. 14, 2005, designating the United States of America, and published, in English, as PCT International Publication No. WO 2005/068087 A2 on Jul. 28, 2005, which application claims priority to European Patent Application Serial No. 04075107.5, filed Jan. 16, 2004, the contents of each of which are hereby incorporated herein by this reference.

### TECHNICAL FIELD

The invention relates to methods for coating objects with hydrophobins. More specifically, the invention relates inter alia to novel methods of stabilizing a hydrophobin coating.

### BACKGROUND

Classically, hydrophobins are a class of small secreted cysteine-rich proteins of fungi or bacteria that assemble into amphipatic layers when confronted with hydrophilic-hydrophobic interfaces. Some hydrophobins form unstable, others extremely stable, amphipatic layers. By assembling at a cell wall-air interface, some have been shown to provide a hydrophobic surface, which has the ultrastructural appearance of rodlets as on aerial hyphae and spores. Some hydrophobins have been shown to assemble into amphipatic layers at interfaces between water and air, water and oils, or water and hydrophobic solids. It appears that hydrophobins are among the most abundantly secreted proteins of fungi, and individual species may contain several genes producing divergent hydrophobins, possibly tailored for specific purposes. Hydrophobins have now been implicated in various developmental processes, such as formation of aerial hyphae, fruit bodies and conidia, and may play essential roles in fungal ecology, including spore dissemination, pathogenesis and symbiosis, and may be involved in adherence phenomena.

Classically known hydrophobins (see, for example, WO 96/41882, which also provides guidance to obtain genetically modified hydrophobin-like substances) typically are proteins with a length of up to 125 amino acids, with a conserved sequence Xₙ—C—X₅₋₉—C—C—X₁₁₋₃₉—C—X₈₋₂₃—C—X₅₋₉—C—C—X₆₋₁₈—C—Xₘ, wherein X represents a n and m independently represent an integer. Most classical hydrophobins contain the above-indicated eight conserved cysteine residues that can form four disulphide bridges. However, when the disulphide bridges of a hydrophobin are reduced by chemical modification and the sulfhydryl groups blocked with, for example, iodoacetamide, the protein assembles in water in the absence of a hydrophilic-hydrophobic interface. The structure is indistinguishable from that of native hydrophobin assembled at the water-air interface. Apparently, the disulphide bridges of hydrophobins keep hydrophobin soluble in water, e.g., within the cell in which they are produced or in the medium, allowing self-assembly at a hydrophilic-hydrophobic interface but they are not necessary to provide for its amphipatic character per se.

All hydrophobins that have been physically isolated thus far self-assemble at hydrophilic-hydrophobic interfaces into amphipatic membranes. One side of the hydrophobin membrane is moderately to highly hydrophilic (with a water contact angle that, for example, ranges between 22° and 63°), while the other side exposes a surface with water contact angles ranging, for example, between 93° and 140°, which indicates as strong a hydrophobicity as, for example, Polytetrafluoroethylene (PTFE or Teflon®) or paraffin (water contact angle at about 110°-120°). The membranes formed by the classical or class I hydrophobins (e.g., those of SC3 and SC4 of Schizophyllum commune) are highly insoluble (e.g., resisting a treatment with 2% sodium dodecyl sulphate (SDS) at 100° C.) but can be dissociated by agents such as formic acid (FA) or trifluoroacetic acid (TFA). In contrast, membranes of the class II hydrophobins cerato-ulmin (CU) of Ophiostoma ulmi and cryparin (CRP) of Cryphonectria parasitica readily dissociate in 60% ethanol and in 2% SDS, while assembled CU is also known to dissociate by applying pressure or by cooling.

Because of the interfacial self-assembly into amphipatic protein layers, hydrophobins can change the wettability of surfaces. As said, one method to measure wettability is by estimating or measuring the contact angle that a water drop makes with the surface. A large contact angle indicates a more hydrophobic surface, a small contact angle, a more hydrophilic surface. Furthermore, in gas/liquid, such as in vigorously shaken water or liquid/liquid systems, such as in oil-in-water or water-in-oil dispersions, air bubbles or oil droplets in solution of hydrophobin become coated with an amphipatic layer that stabilizes them. Solid/liquid interfaces show the same stabilization. For example, a sheet of hydrophobic plastic such as PTFE immersed in hydrophobin solution becomes coated with a strongly adhering protein layer that makes the surface completely wettable (contact angle 40-55°), even after hot SDS treatment, and hydrophobins attached on a hydrophilic surface make the surface less hydrophilic, or even more hydrophobic.

Self-assembly of hydrophobins is accompanied by conformational changes (De Vocht et al. (1998), Structural characterization of the hydrophobin SC3, as a monomer and after self-assembly at hydrophobic/hydrophilic interfaces, Biophysical Journal 74:2059-2068). Monomeric class I hydrophobins are rich in β-strands. At the water-air interface, class I hydrophobins more easily show an increase in β-sheet structure (called the β-sheet state), while at the interface between water and hydrophobic solid, a form with an increased number of α-helices is observed (the α-helical state or α-state). However, this α-helical layer of class II hydrophobins can be rapidly rinsed off. The α-helical state seems to be an intermediate of self-assembly, whereas the β-sheet state is likely to be the stable end-form.

At the water-air interface, dependent on the conditions, monomers of class I hydrophobins attain the α-helical state within seconds, but the conversion to the β-sheet state is much slower and takes minutes. At the water-solid interface, a hydrophobin layer is formed wherein the protein also readily attains the α-helical state. Subsequently, the α-helical state can undergo a transition into a stable β-sheet end state, typically upon exposure of the hydrophobin layer to an increased temperature, e.g., to a temperature of 30° C. or higher (see, PCT International Patent Publication WO 01/57528, the contents of which are incorporated by this reference), in the presence of detergent. The heat treatments are generally performed at a pH of around 7. Detergent has been used in combination with heat as long as hydrophobins are known. Presumably, a high temperature leads to destabilization and flexibility of the hydrophobin molecules that are present in a coating. This destabilization contributes to achieving the conformational rearrangement that leads to strong insoluble coatings. The higher the temperature, the faster the transformation takes place. For example, an object is contacted with a hydrophobin-containing solution, such that a hydrophobin layer is formed at the surface of the object. The temperature is then raised to 60° C. or even higher, such as to 80° C., after which a detergent is added. Thus, heat treatment can be used to enhance the formation of a stable hydrophobin coating on the surface of an object. However, from an economical and practical point of view, a heat treatment can be very unattractive. For example, in case the object to be coated is large (e.g., the hull of a ship) or sensitive to heat, heating the object to be coated to increased temperatures, typically around 80° C., is not desirable.

### DISCLOSURE OF THE INVENTION

Therefore, the present inventors set out to find conditions other than increasing the temperature that can be used to obtain a stable hydrophobin coating on the surface of an object. Surprisingly, it was discovered that the transformation of an unstable hydrophobin layer into a stable coating can also be obtained at a low temperature (e.g., below 30° C.) in the presence of detergent at a low pH and in the absence of detergent at a low pH, a high concentration of hydrophobin, prolonged incubation and/or the presence of a buffer. Therefore, the invention relates to a method for optimizing the conditions for providing the surface of an object with a hydrophobin coating by contacting at least part of the surface with a hydrophobin-containing solution at room temperature, comprising determining the effect of at least two parameters on the formation of a hydrophobin layer in the surface of the object, wherein the parameters are selected from the group consisting of pH, incubation time, concentration of hydrophobin in the hydrophobin-containing solution and presence of a buffer in the solution.

The effect of parameters on the formation of a hydrophobin layer on the surface of an object can be determined by various means. These include contact angle measurements and circular dichroism (CD) spectroscopy (see Examples below).

In a first aspect, the invention provides the insight that a low pH can promote the stabilization of hydrophobin layers. Accordingly, the invention provides a method for providing the surface of an object with a hydrophobin coating, comprising contacting at least a part of an object with a hydrophobin-containing solution to form a hydrophobia layer on the surface of the object and exposing the layer to a pH below 7, preferably below 4, more preferably below 2, optionally in the presence of a detergent.

Many different kind of objects, or at least a part thereof, may be coated using a method of the invention. Examples are: a glass surface such as a window, a contact lens, a biosensor, a medical device, a container for performing an assay or storage, the hull of a vessel or a frame or bodywork of a car, a solid particle, a textile (e.g., clothing), a porous object or material and the like.

From an economical point of view, a method provided herein can be very advantageous as it is often easier to lower the pH than to heat to increased temperatures. Hot detergent treatments known in the art to enhance the formation of a stable hydrophobin coating have resulted in 10-30% loss of hydrophobin from the surface, which was accompanied with the appearance of pores in the coating. Janssen et al. (2003) (“Promotion of fibroblast activity by coating with hydrophobins in the β sheet end state,” M. I. Janssen, M. B. M. van Leeuwen, T. G. van Kooten, J. de Vries, L. Dijkhuizen and H. A. B. Wösten, Biomaterials, June. 2004; 25(14):2731) reported that α-helical SC3 forms a uniform layer, whereas the β-sheet SC3 coating induced by increased temperature was discontinuous. It is conceivable that a low pH treatment is milder than a hot detergent treatment.

### BRIEF DESCRIPTION OF THE DRAWINGS

FIG. 1: The presence of polyvinyl alcohol (PVOH) in the culture medium prevents assembly of SC3. The total amount of SC3 produced was determined by TCA precipitation of medium from cultures without PVOH (lane 1), containing 0.1% PVOH (lane 2) or containing 0.3% PVOH (lane 3). The culture media without PVOH or containing either 0.1% or 0.3% PVOH were vortexed (two minutes, maximum speed) and centrifuged (15 minutes 13,000 rpm). The pellet fractions (lanes 4 to 6, respectively) and 10% TCA precipitated supernatant fractions (lanes 7 to 9, respectively) were analyzed separately.

FIG. 2: Circular dichroism (CD) spectra of SC3 showing the transition from soluble state (orange) to β-sheet state (blue lines) at room temperature in the presence of 1% PVOH. The red line indicates that at pH 7 no change occurs in a time course of hours, whereas at pH 4, a β-sheet has formed.

FIG. 3: The Thioflavin T fluorescence at each SC3 concentration is plotted and fitted with a straight line.

FIG. 4: Circular dichroism (CD) experiments were performed with PFA-SC3. Sufficient PFA-SC3 was used to keep the signal between −10 and −40 in a 1 mm cuvette. PFA-SC3 in water shows a spectrum most comparable with an unstructured peptide (dark grey). Upon addition of colloidal PTFE, the spectrum changes instantly to an α-helical state structure that is completely identical to that of SC3 bound to PTFE (light grey).

FIG. 5: Calcium can-be used to stabilize a PFA-SC3 layer, as monitored using CD spectroscopy.

FIG. 6A: Contact angles of PFTE sheets coated in hydrophobin solutions at a concentration of 300, 100, 50 or 5 μg/ml in 50 mM phosphate buffer pH 7 during 16 hours, 3 hours or 15 minutes. Samples were washed with milliQ water or with 0.1% TWEEN 20 pH 7 (Tw7).

FIG. 6B: Contact angles of PFTE sheets coated in hydrophobin solutions at a concentration of 300, 100, 50 or 5 μg/ml in 50 mM phosphate buffer pH 7 during 16 hours, 3 hours or 15 minutes. Samples were incubated in 1% SDS at 100° C. for 10 minutes. Samples were washed with milliQ water or with 0.1% TWEEN 20 pH 7 (Tw7).

FIG. 7: Contact angles of PFTE sheets coated in hydrophobins solubilized in water at pH 4 or in water at pH 7 at a concentration of 300, 100 or 50 μg/ml. Coating was performed during 16 hours or 3 hours. Samples were washed with milliQ water or with 0.1% TWEEN 20 pH 7 (Tw7).

FIG. 8: Contact angles of PFTE sheets coated in hydrophobins solubilized in milliQ water pH 7 or in 50 mM phosphate buffer pH 7 at a concentration of 300, 100 or 50 μg/ml. Coating was performed during 16 hours or 3 hours. Samples were washed with milliQ water or with 0.1% TWEEN 20 pH 7 (Tw7).

### DETAILED DESCRIPTION OF THE INVENTION

The term “hydrophobin,” as used herein, refers not only to the classical hydrophobins as defined above, but also comprises essentially amphiphatic proteins capable of coating a surface, rendering a hydrophobic surface essentially hydrophilic or, vice versa, a hydrophilic surface essentially hydrophobic, and comprises not only hydrophobins that can be isolated from nature but includes substances that can be obtained by genetically modifying genes to obtain genetically modified proteins not at present available from nature, still having or having obtained the desired amphipatic characteristics.

The term “hydrophobin layer” is to be understood as an amphipatic hydrophobin layer or membrane which essentially has the characteristics of hydrophobins in the α-helical conformation, i.e., it can be solubilized by detergent at room temperature and neutral pH.

The term “hydrophobin coating” refers to an amphipatic hydrophobin membrane with the characteristics of hydrophobins in the β-sheet conformation, i.e., it is stable or resistant against a treatment with a detergent at room temperature. Without wishing to be bound by theory, a low pH of a solution surrounding hydrophobin may cause a similar destabilization of hydrophobin as increased temperatures. It has been described for amyloid peptides that fibrils are induced solely by decreasing the pH or by increasing the salt concentration. These conditions have a destabilizing effect on the soluble amyloid peptides which then assembles in fibril structures. Detergent is not needed for fibril formation for amyloid molecules. Moreover, the process takes place solely in water. Hydrophobin activity is associated with interfaces, preferably hydrophilic/hydrophobic, such as air/water, oil/water and solid/water. Hydrophobins share many properties with other amyloid proteins (Butko et al., 2001, Spectroscopic evidence for amyloid-like interfacial self-assembly of hydrophobin SC3, Biochem. Biophys. Res. Commun. 280:212-215; Wösten and de Vocht 2000, Hydrophobins, the fungal coat unraveled, Biochim. Biophys. Acta 1469:79-86; McKay et al. 2001, The hydrophobin EAS is largely unstructured in solutions and functions by forming amyloid-like structures, Structure 9:83-91), although similarities on an amino acid level are not significant.

According to the invention, the transition to the β-state can be affected by exposing a hydrophobin layer on the surface of an object or a part thereof to a pH below 7, preferably below 4, more preferably below 2. This can be performed at around room temperature. For example, a stable coating can be formed at a low pH at a temperature below 30° C., for example, at a temperature of only 25° C., 20° C. or 15° C. or even lower. Temperatures as low as 5° C. or even lower may be used provided that, if a detergent is used, the detergent is still effective. Generally speaking, a temperature of around room or ambient temperature is, of course, most practical for many applications because this does not require any heating or cooling.

Another major advantage of the low pH-induced assembly is that it may be performed as one single step. In contrast, if elevated temperatures are used in combination with detergent, the detergent can only be added once the temperature is higher, otherwise the hydrophobin is washed away. Surprisingly, lowering the pH, optionally while detergent is present, is sufficient to result in stable hydrophobin coating.

The hydrophobin transition from the α-helix state to the insoluble β-sheet state on an air-water interface takes place without detergent. For a similar (i.e., equally fast) transition on the surface of an object, the presence of a detergent is required, probably for making the hydrophobin molecules mobile on this surface until the transition takes place, after which the hydrophobin cannot be dissolved by detergent any more. As is exemplified herein, the rate of transformation into a β-state is dependent on both the temperature and pH. At pH 2, a stable coating of SC3 is formed on PTFE within 30 minutes at 15° C., whereas at pH 2, the coating instantly forms at 25° C. or higher (see Table 1).

Hydrophobins are among the most potent biosurfactants known and are able to modify surface properties of solids and stabilize gas bubbles and oil droplets in water. They form amphipatic layers and adhere efficiently to both natural and man-made surfaces. PTFE, for example, can be coated by a very stable hydrophobin layer, and only a few milligrams are needed per square meter. Upon aggregation, highly ordered structures, fibrils and films, are formed. Due to the dual properties, surface activity and self-assembly, hydrophobins are highly interesting for many different applications. For example, it has been suggested to coat a surface of, for example, a biosensor, with a hydrophobin to modify the hydrophobic/hydrophilic nature of the surface. A hydrophobin-containing solution should be handled with care, as actions such as shaking result in turbid solutions containing hydrophobin aggregates, which affect a uniform coating of a surface. Furthermore, for the application of a hydrophobin on a significant scale, an industrial scale method is necessary for purifying a hydrophobin present in a hydrophobin-containing solution, such as growth medium of a fermentation culture. A method according to the state of the art relies on the use of TFA, which is not desirable for environmental and safety reasons. More importantly, whereas production of a hydrophobin (e.g., SC3) in the growth or culture medium of an organism producing the hydrophobin. (e.g., S. commune) can be as high as 50 mg per liter or even higher, known purification schemes can lead to losses of up to 90%. Typically, assembled hydrophobins are isolated from the culture medium by bubbling or centrifugation. The most inefficient step in the purification schemes is extracting or solubilizing hydrophobin from this “primary hydrophobin pellet” with TFA.

Surprisingly, as a further aspect of the invention, it has now been found that purification of hydrophobin from the culture medium is no longer required if hydrophobin is directly used as a coating substance, since the hydrophobin concentration of the culture medium is usually sufficiently high to use the culture medium directly. The choice of production organism can, of course, influence the dominance of the hydrophobin among the secreted proteins.

In one aspect of the invention, a method for providing the surface of an object with a hydrophobin coating is provided, comprising contacting at least a part of an object with the supernatant of a culture medium (culture supernatant) of an organism that secretes a hydrophobin at a pH below 7, preferably below 4, more preferably below 2, optionally in the presence of a detergent.

A supernatant, also referred to as culture supernatant or coating solution, as used herein is derived from a liquid culture or growth medium that has been used during a certain period of time to culture or grow an organism that produces and secretes hydrophobin into the medium such that the medium contains a certain amount of a hydrophobin. Following a certain culturing period, the culture supernatant is typically prepared by separating the culture medium (containing the hydrophobin) from the organism, e.g., by filtering the medium over a cloth. Furthermore, compounds or contaminants may be removed from the culture supernatant prior to contacting it with a surface of an object to be coated, for instance, by dialysis.

The culture medium of different types of hydrophobin-secreting organisms are suitably used in a method according to the invention. Preferably, the organism is a fungus, more preferably, the basidiomycete fungus Schizophyllum commune. Other suitable organisms include: Agaricus bisporus, Pleurotus ostreatus, Coprinus cinereus, Lentinula edodes, Agrocybe aegerita, Pisolithus tinctorius, Ustilago maydis, Magnaporthe grisea, Aspergillus nidulans, Aspergillus fumigatus, Metarhizium anisopliae, Xanthoria ectaneoides, Xanthoria parietina, Cladosporium fulvum, Neurospora crassa, and strains that are (genetically) engineered to produce hydrophobins.

To ensure that a sufficient amount of hydrophobin assembles on the surface of an object to form a hydrophobin layer, the hydrophobin content of the culture supernatant should be at least 2 mg hydrophobin per liter, preferably at least 5 mg/l, more preferably at least 10 mg/l, or even higher.

In a preferred embodiment, an object is contacted with a hydrophobin-containing culture supernatant comprising a detergent at a low pH such that a hydrophobin coating is formed on the surface of the object. The detergent may be added to the culture medium or supernatant after completion of the culturing period, for example, prior to harvesting. Preferably, however, the detergent is present in the culture medium during culturing of the organism such that self-assembly of hydrophobins is prevented once they are secreted into the medium. To this end, a detergent may be added to the culture medium prior to or during culturing of the organism. Alternatively, a hydrophobin-producing organism may produce and secrete a compound with a detergent-like function, such that no exogenous detergent needs to be added. The presence of a detergent during culturing is particularly advantageous when agitating cultures are used (as opposed to standing cultures) for the production of hydrophobins, since it is known that agitation normally causes assembly of hydrophobins and, therefore, renders the protein or the medium containing the protein unsuitable for coatings. In a method of the invention, the presence of a detergent prevents self-assembly of hydrophobins in the culture supernatant.

A detergent molecule is characterized by a hydrophilic “head” region and a hydrophobic “tail” region. The result of this characteristic is the formation of thermodynamically stable micelles with hydrophobic cores in aqueous media. This hydrophobic core provides an environment that allows for the dissolution of hydrophobic molecules or domains of proteins. Detergents are also called amphiphiles or surfactants. “Surfactant” is short for “SURFace ACTive AgeNT,” a molecule that lowers surface tension. These molecules contain both hydrophobic and hydrophilic components and are thus semisoluble in both organic and aqueous solvents.

Different types of detergents may be added to the culture supernatant. Examples of detergents are APO-10, APO-12, BRIJ-35 (C12E23), C8E6, C10E6, C10E8, C12E6, C12E8 (Atlas G2127), C12E9, C12E10 (Brij 36T), C16E12, C16E21, Cyclohexyl-n-ethyl-β-D-Maltoside, Cyclohexyl-n-hexyl-β-D-Maltoside, Cyclohexyl-n-methyl-β-D-Maltoside, n-Decanoylsucrose, n-Decyl-β-D-glucopyranoside, n-Decyl-β-D-maltopyranoside, n-Decyl-β-D-thiomaltoside, Digitonin, n-Dodecanoyl sucrose, n-Dodecyl-β-D-glucopyranoside, n-Dodecyl-β-D-maltoside, Genapol C-100, Genapol X-80, Genapol X-100, HECAMEG, Heptane-1,2,3-triol, n-Heptyl-β-D-glucopyranoside, n-Heptyl-β-D-thioglucopyranoside, LUBROL PX, MEGA-8 (Ocatanoyl-N-methylglucamide), MEGA-9 (Nonanoyl-N-methylglucamide), MEGA-10 (Decanoyl-N-methylglucamide), n-nonyl-β-D-glucopyranoside, Nonidet P-10 (NP-10), Nonidet P-40 (NP-40), n-Octanoyl-β-D-glucoslyamine (NOGA), n-Octanoyl sucrose, n-Octyl-alpha-D-glucopyranoside, n-Octyl-β-D-glucopyranoside, n-Octyl-β-D-maltopyranoside, PLURONIC F-68, PLURONIC F-127, THESIT, TRITON X-100 (tert-C8-Ø-E9.6; like NP-40), TRITON X-100 hydrogenated, TRITON X-114 (tert-C8-Ø-E7-8), TWEEN 20 (C12-sorbitan-E20; Polysorbate 20), TWEEN 40 (C16-sorbitan-E20), TWEEN 60 (C18-sorbitan-E20), TWEEN 80 (C18:1-sorbitan-E20) and n-Undecyl-β-D-maltoside. Representative examples of long chain or high molecular weight (>MW 1000) detergents include gelatin, casein, lecithin (phosphatides), gum acacia, cholesterol, tragacanth, polyoxyethylene alkyl ethers, polyoxyethylene castor oil derivatives, polyoxyethylene sorbitan fatty acid esters, e.g., the commercially available TWEENs, polyethylene glycols, polyoxyethylene stearates, colloidal silicon dioxide, phosphates, sodium dodecylsulfate (SDS), carboxymethylcellulose calcium, carboxymethylcellulose sodium, methylcellulose, hydroxyethylcellulose, hydroxypropylcellulose, hydroxypropylmethylcellulose phthalate, microcrystalline cellulose, magnesium aluminum silicate, triethanolamine, polyvinyl alcohol (PVOH), and polyvinylpyrrolidene (PVP). Low molecular weight (MW <1000) detergents include stearic acid, benzalkonium chloride, calcium stearate, glycerol monostearate, cetostearyl alcohol, cetomacrogol emulsifying wax, and sorbitan esters.

According to the invention, a detergent or surfactant can be present in a concentration of at least 0.001% wt./vol., preferably at least 0.01% wt./vol., more preferably at least 0.1% wt./vol. and with the highest preference at least 1% wt./vol., depending on the type of detergent, the organism and the culture conditions.

Of course, in accordance with the present invention, it is most appropriate if an organism is cultured in the presence of a detergent that is metabolized or degraded by the organism to only a small extent, preferably not at all. If a detergent is metabolized during the culturing period, this would require replenishment of the detergent during the culturing period. In addition, the detergent preferably does not have an inhibitory effect on the growth of the organism and/or the production and secretion of hydrophobin. Needless to say, the detergent should also be capable (in combination with an elevated temperature or a reduced pH, as will be discussed below) of stabilizing a hydrophobin layer such that a coating is formed. Suitable are those detergents which are commonly used in methods for providing a hydrophobin coating on the surface of an object, such as Triton X100, PVOH, TWEEN 20, TWEEN 80 and SDS.

In the presence of detergent, hydrophobin will not adhere to the PFTE surface. Therefore, a change in contact angle can be directly related to the amount of protein adhered to the surface (see, e.g., Examples 2 and 4). However, in the absence of detergent, there is no direct correlation of contact angle and amount of hydrophobin adhered. Therefore, the quality of coating in the absence of detergent can be measured by determining the change in contact angle and resistance upon washing with detergent (e.g., 0.1% TWEEN 20 pH 7; see Examples 10-12), as indicated by contact angle, without quantification of the amount of hydrophobins adhered to the surface.

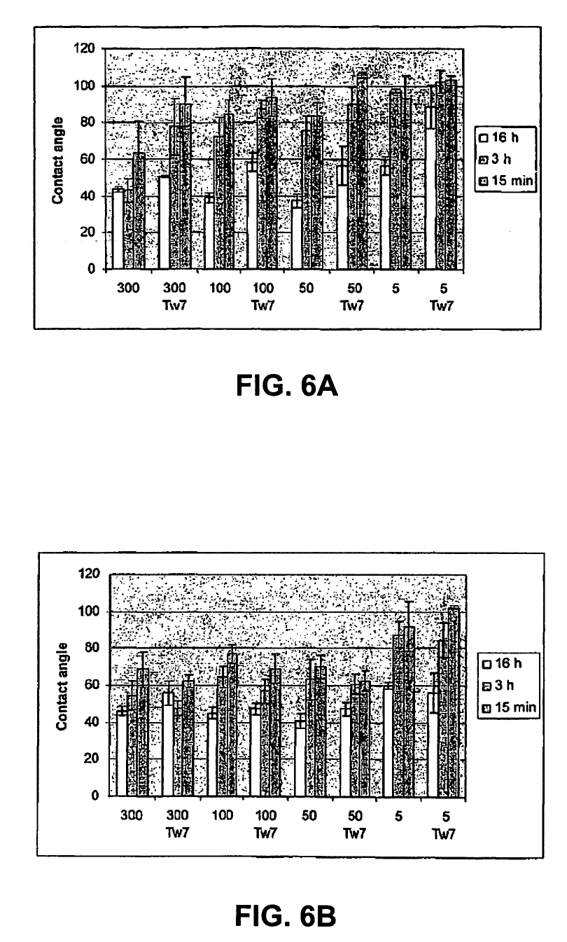

In search for conditions that enhance the formation of a hydrophobin coating at mild or low temperatures, it was surprisingly found that the concentration of hydrophobin in the coating solution is also a factor that affects the formation of a hydrophobin coating on the surface of an object; the higher the concentration used, the faster a coating is formed. For example, coating of PTFE sheets with a solution of 300 μg/ml hydrophobin in sodium phosphate buffer (pH 7.0) during 16 hours at room temperature in the absence of a detergent resulted in a coating with a low water contact angle (around 45 degrees; see Example 10). When a concentration of only 5 μg/ml was used, a 16-hour incubation period yielded a contact angle of around 55 degrees. However, this sample appeared to be unstable because the contact angle increased to around 90 degrees upon exposure to TWEEN 20 at pH 7. An incubation of three hours at 300 μg/ml resulted in a contact angle of around 55 degrees, which is similar to the contact angle of the 16-hour incubation at the same concentration. However, unlike the 16-hour incubation, this three-hour incubation resulted in an unstable sample because the contact angle increased to around 80 degrees upon exposure to TWEEN 20 at pH 7. These data show that a coating can be obtained when using a high concentration of hydrophobin (e.g., 300 μg/ml) in the coating solution and a prolonged incubation time (e.g., 16 hours). In addition, the concentration of hydrophobin also affected the obtained contact angles. For example, coating of PTFE sheets with a solution of 300 μg/ml hydrophobin in sodium phosphate buffer (pH 7.0) during three hours at room temperature resulted in a low water contact angle (around 45 degrees; see Example 10). When a concentration of 50 μg/ml was used, three hours of incubation yielded a contact angle of around 70 degrees. In one embodiment, the invention provides a method for providing the surface of an object with a hydrophobin coating, comprising contacting at least a part of an object with a hydrophobin-containing solution to form a hydrophobin coating on the surface of the object, wherein contacting is performed during 16 hours or more at a temperature below 30° C. (e.g., at room temperature) using a concentration of more than 50 μg/ml hydrophobin, preferably more than 100 μg/ml, more preferably more than 300 μg/ml. For example, an object is contacted at 25° C. with a hydrophobin solution that contains 300 μg/ml hydrophobin (e.g., SC3) for 16 hours to provide the surface of the object with a hydrophobin coating. As is clear from FIG. 6A, a longer incubation period results in a lower contact angle and an increased stability (as evidenced by the ability to wash off the hydrophobins with TWEEN 20) of the sample.

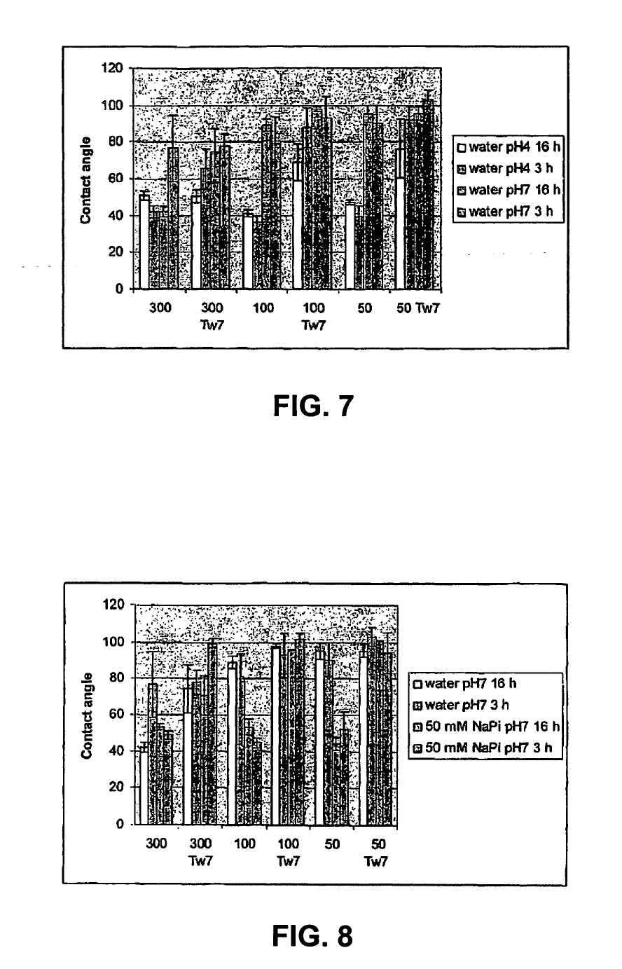

A similar time and concentration dependency was found when hydrophobin solutions were prepared in either water pH 4 or in water p H7 (see Example 11 and FIG. 7). Moreover, lowering the pH of the coating solution from 7 to 4 resulted in (for each concentration tested) reduced contact angles and resulted in enhanced stability of the samples at high hydrophobin concentrations.

Next, the effect of buffer on the coating efficiency was determined. Hydrophobin solutions (50, 100 or 300 μg/ml) prepared in either water pH 7 or 50 mM sodium phosphate pH 7 were used to coat PTFE sheets at room temperature during 3 or 16 hours (see Example 12). The results (FIG. 8) clearly demonstrate that addition of buffer to the coating solution affects the obtained contact angles but does not affect the stability of the samples. The presence of buffer decreased contact angles at all concentrations tested following the three-hour incubation period. Buffer also influenced the coatings obtained after 16 hours using 50 or 100 μ/ml hydrophobin. The contact angle obtained after 16 hours with a 300 μg/ml hydrophobin solution prepared in water pH 7 could not be further reduced upon addition of buffer. Apparently at this point, the maximally obtainable result is already achieved and modification of additional parameters will not further enhance coating efficiency.

Herewith, the invention provides a method for providing the surface of an-object with a hydrophobin coating, comprising contacting at least a part of an object with a hydrophobin-containing solution to form a hydrophobin layer on the surface of the object, wherein the hydrophobin-containing solution comprises a buffer. A buffer is an ionic compound that resists changes in its pH. A buffer solution is a mixture of a weak acid HA and its conjugate base A− (usually added under the form of the sodium or potassium salt, NaA or KA). Alternatively, a mixture of a weak base B and of its conjugate acid BH+ is also a buffer solution. In one embodiment, a hydrophobin solution is made in a phosphate buffer, preferably a sodium phosphate (NaPi) buffer, for example, a 25 or 50 mM NaPi buffer. The pH of the buffer solution can vary. Preferably, contacting with a buffered hydrophobin solution is performed at a temperature below 30° C. In one embodiment, the concentration hydrophobin in the solution is at least 5, preferably at least 20, more preferably at least 50, even more preferably at least 100 μg/ml, and most preferably at least 300 μg/ml.

The invention demonstrates that various parameters affect the formation of a stable coating at mild temperatures: pH, incubation time, hydrophobin concentration and the presence of a buffer and/or detergent. As shown herein by the numerous examples, any one or combination of parameters can be used to optimize coating conditions. As a general rule of thumb, the rate at which a hydrophobin coating is formed is enhanced by a low pH (e.g., pH 4 or 2), with or without the presence of detergent (e.g., 0.1% TWEEN 20), and a high concentration of hydrophobin. Of course, these technical measures may be combined to yield optimal coating conditions for a particular situation. For instance, if an object is to be coated at room temperature in a minimal period of time, a highly concentrated solution of 500 μg/ml or more may be used, optionally in combination with a low pH with or without detergent. Of course, the larger the surface to be coated, the more (purified) hydrophobia is required and it should be noted that the concentration used is also dependent on the surface area to be coated. Therefore, for the coating of large surfaces, highly concentrated coating solutions are economically less attractive. In these cases, lower hydrophobin concentrations are typically used that require longer incubation times. For example, a culture supernatant is advantageously used as a coating solution. As shown herein, the incubation times can be reduced upon lowering the pH, optionally together with the addition of a detergent.

In a specific aspect of the invention, the coating of the surface of at least part of an object is performed by contacting the surface with a hydrophobin-containing solution wherein the solution further comprises one or more additives such that upon the formation of the hydrophobin coating, the additive(s) become(s) incorporated in the coating. Exemplary additives include biologically active compounds of natural or synthetic origin (peptide hormones, drugs or other therapeutic molecules). The incorporation may be reversible, allowing for a slow release of the additive out of the coating into its surroundings. In one embodiment, the object to be coated with a hydrophobin layer comprising an additive is a medical device. For instance, a catheter is contacted with a solution that contains hydrophobin and a drug to provide a catheter that slowly releases the drug. Any means known in the prior art and disclosed herein may be used to enhance the formation of the coating at the surface, including lowering the pH and addition of detergent.

Conventional procedures for purification of hydrophobins rely on the use of TFA. Instead of TFA, performic acid PFA can be used to dissolve assembled hydrophobin. This was described by de Vries et al. (1993; Insoluble hydrophobin complexes in the walls of Schizophyllum commune and other filamentous fungi, Arch. Microbiol. 159:330-335), who used PFA to dissolve insoluble SC3 in order to analyze it on SDS-PAGE. It is now revealed that PFA-treated hydrophobin is advantageously used to maintain hydrophobin in a soluble state in a hydrophobin-containing solution. It was found that PFA-SC3 forms nice α-helical structures on colloidal PTFE when observed by circular dichroism (CD) spectroscopy. Similar observations have been made by de Vocht et al. (2000 Structural and functional role of the disulfide bridges in the hydrophobin SC3, J. Biol. Chem. 275:28428-28432) when SC3 was chemically modified by iodoacetic acid.

Thus, in one embodiment, the invention provides a method for coating the surface of an object with a hydrophobin coating, comprising contacting an object with a hydrophobin-containing solution, wherein the solution comprises hydrophobin that has been treated with PFA. PFA-treated hydrophobin is preferably freeze-dried (lyophilized) hydrophobin, preferably freeze-dried purified hydrophobin, which has been dissolved in a PFA solution. PFA treatment is thought to oxidize the cysteines, and disulfide bonds of hydrophobins to sulfonates. The formation of correct disulfide bonds is important for a good assembly of hydrophobins. The treatment of hydrophobins with PFA could help in solubilizing aggregated hydrophobins and/or functionalize inactive hydrophobins, for instance, recombinantly produced heterologous hydrophobins, including mutant hydrophobins. Inactive hydrophobins include hydrophobins with no or randomly formed disulfide bonds.

In accordance with the aforementioned procedure, it was observed that exposure of a layer of PFA-treated hydrophobin to a pH below 7, preferably below 4, more preferably below 2, can be used to stabilize the hydrophobin layer such that a firm coating is obtained.

Furthermore, it was found that PFA-SC3 interacts specifically with divalent metal ions and that a stable hydrophobin coating cannot only be formed by a low pH but also by exposing a PFA-SC3 to divalent metal ions. Upon this interaction, a clear structural change is observed in solution upon mixing PFA-SC3 with bivalent metal ions, for example, Ca²⁺. Moreover, when colloidal PTFE was added to this mixture, no α-helical state was observed (or only transiently) but the hydrophobin layer appeared to be in a state very similar to the β-sheet coating state. This was observed in the absence of detergent and at room temperature. Apparently, the performic acid derivatives of cysteine can be paired up by addition of calcium such that a disulfide-like bond is formed. PFA treatment of hydrophobins as provided herein has several advantages. First, there is no need for the highly toxic TFA to solubilize hydrophobin. Second, PFA-treated hydrophobins do not aggregate upon agitation. Importantly, they can still form a β-sheet (as observed with CD) under the right conditions, i.e., with addition of metal ions or lowering pH.

The invention is further described with the aid of the following illustrative examples.

### EXAMPLES

### Example 1

### Soluble State of SC3 in Culture Supernatant of S. commune Containing the Detergent PVOH

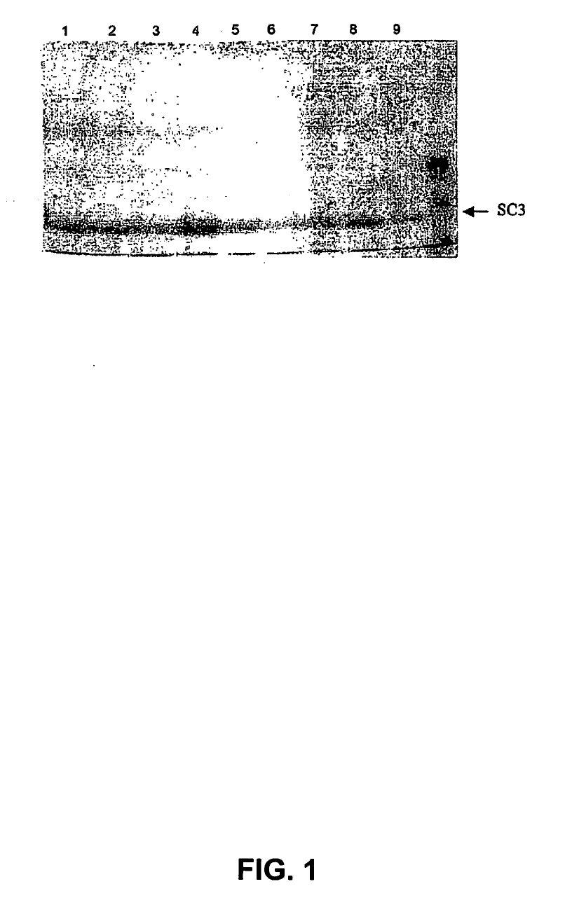

200 ml cultures of S. commune (ΔSC15) were inoculated by fragmenting one-half of a colony in 40 ml production medium (PM) using a WARING blender. Two ml of the fragmented material (macerate) was added to 200 ml PM in a 1 L Erlenmeyer flask. Similarly, two cultures with 200 ml PM containing either 0.1 or 0.3% PVOH were inoculated. The cultures were grown at 30° C. and 200 rpm. The cultures were harvested at a glucose concentration of below 5 g/L. The culture supernatant was obtained by separating the medium from the mycelium by filtering over a nylon cloth. Analysis of the production levels of the hydrophobin SC3 by SDS-PAGE and Coomassie-staining of 450 μl 10% TCA precipitated medium showed that the amount of SC3 produced in the culture containing 0.1% PVOH was similar to that in the cultures grown without PVOH (FIG. 1, lanes 1 and 2). In the 0.3% PVOH-containing culture, SC3 production was slightly decreased (FIG. 1, lane 3).

450 μl of the different culture supernatant samples were vortexed for two minutes at maximum speed and centrifuged for 15 minutes at 13,000 rpm. Analysis of the pellet and 10% TCA precipitated supernatant fractions by SDS-PAGE showed that in the culture supernatant containing 0.1% PVOH, a major part of the SC3 remained in the supernatant fraction (FIG. 1, lane 5) compared to the pellet fraction (FIG. 1, lane 8). This effect was even more pronounced in the culture supernatant containing 0.3% PVOH (FIG. 1, lane 6 and 9). Analysis of the culture supernatant without PVOH showed that the SC3 was mainly present in the pellet fraction (FIG. 1, lane 4) when compared to the supernatant fraction (FIG. 1, lane 7). Thus, the presence of PVOH in the culture medium prevents hydrophobin assembly on air-water interfaces (applied by vortexing).

### Example 2

### Coating with Culture Supernatant—Contact Angles of PTFE-Sheets Coated in Culture Supernatant Containing PVOH

PTFE (polytetrafluoroethylene; PTFE) sheets of 2 cm² were thoroughly cleaned with 100% ethanol, pure TFA and washed with water. PTFE sheets were placed in 2 ml containers containing 2 ml of culture supernatant without PVOH and supernatant with 0.1%-2% PVOH present during growth, such that a layer was formed on the surface of the sheet. The supernatants were obtained as described in Example 1. A hydrophobin layer was prepared by incubation for 16 hours at 25° C. in supernatant (pH 5.5) or in supernatant that was acidified to pH 2 with TFA or HCl. The coated sheets were washed three times for five minutes with milliQ water and were left to dry. The hydrophilicity of the coated surface was determined with a Drop Shape Analysis System DSA 10 Mk2 apparatus (Krüss) by measuring the contact angle of 1-2 μl milliQ water with the surface.

The results show that culture supernatant of shaken cultures (with or without added PVOH) that is directly used for coatings yield high contact angles of above 85°, which is only a small decrease when compared to the contact angle of the PTFE sheets of typically 110°. Acidifying the medium with HCl or TFA to a pH of 2 prior to coating resulted in significant low contact angles of the PTFE sheets for both culture medium of cultures grown in the presence or absence of PVOH. Contact angles of 50° to 60° could be obtained routinely for acidified medium of normal and detergent-containing cultures. These coatings contain SC3 hydrophobin that is secreted in the culture medium as is confirmed by antibody detection and SDS-PAGE. Coatings with contact angles of as low as 30° could be obtained with acidified medium of cultures that were grown in the presence of 0.1% PVOH. Higher concentrations of PVOH did not interfere with growth of S. commune, but resulted in coatings with high contact angles. The coating potential of the culture supernatant varied with different growth conditions and with the time of harvesting the culture medium.

The presence of a detergent during growth guaranteed the most reproducible results. However, supernatants without pre-added detergent could also be useful for coating purposes. This indicates that detergent-like substances are secreted by the fungus that help to keep the secreted SC3 in a state that is suitable for coating. In all cases, the pH of the medium supernatant was lowered in order to obtain good coatings.

Coating experiments were also performed with glass slides that were cleaned by boiling them for 20 minutes in a 2% SDS solution followed by extensive rinsing with water. Coatings were performed as described for PTFE sheets. The contact angles of uncoated glass slides were undetectable and are, therefore, lower than 15°. Coating of glass slides with culture supernatant with or without added detergent resulted in contact angles of 20° to 40°, presumably reflecting the formation of a layer of hydrophobin on the glass surface. Acidifying the medium prior to coating resulted in contact angles of 50° to 65°, which is an increase of 50°, making the glass more hydrophobic.

### Example 3

### Coating an Object with Hydrophobin by Exposure to a Low pH in the Presence of a Detergent CD Experiments, Varying Conditions, Detergent and Time Course

The secondary structure of SC3 was studied with circular dichroism spectroscopy (CD). The CD spectra were recorded over the wavelength of 190-250 nm on an Aviv 62A DS CD spectrometer (Aviv Associates, Lakewood, N.J., USA), using a 1-mm quartz cuvette. The spectra were recorded using a reference solution without protein. Typically, a protein concentration of 100 to 200 μg/ml was used. For spectra of hydrophobin bound to a hydrophobic support, 159 nm non-stabilized, colloidal polytetrafluoroethylene (PTFE) in water was added to the solution. Surface coverage of hydrophobin on PTFE was typically 10%. As detergent, 1% polyvinyl alcohol (w/w; PVOH, 88% hydrolyzed) or 0.1% TWEEN 20 were used as a final concentration.

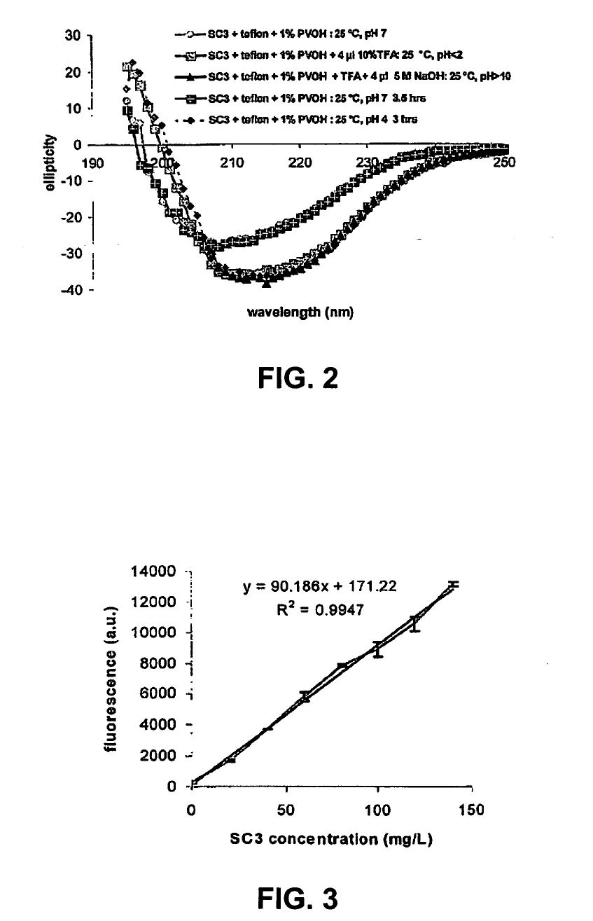

A) 100 μl SC3 (0.5 mg/ml) in 25 mM phosphate buffer (pH 7.0), 200 μl 2% PVOH (w/w), and 100 μl colloidal PTFE were mixed in a quartz cuvette. The solution was equilibrated at 25° C. The CD spectrum was recorded directly and after three hours (FIG. 2). The spectrum remained unchanged and is that of soluble SC3 (de Vocht et al., 1998). The same mixture was made again but after recording a spectrum, 4 μl of 10% trifluoroacetic acid (TFA) was added to drop the pH below 2, mixed and a spectrum was recorded (FIG. 2). The CD spectrum changed from a soluble SC3 spectrum to a completely β-sheet state spectrum. The low pH of the hydrophobin-containing solution had induced the complete transition from soluble SC3 to an insoluble coating on PTFE. Next, the pH within the cuvette was raised above 10 by adding 4 μl of 5 M NaOH. The newly recorded spectrum showed no change compared to the spectrum recorded at low pH (FIG. 2). Therefore, it can be concluded that a temporary decrease in pH irreversibly induces β-sheet state conformation. Thus, SC3 remains soluble in the presence of detergent and colloidal PTFE at pH 7 on a time scale of hours, but forms an insoluble β-sheet at low pH.

B) A similar experiment as performed under A was done with 0.1% TWEEN 20. The results were identical. Thus, a detergent other than PVOH can also be used. SDS, which is normally used for coatings, could not be tested with CD due to strong interference with the signal.

C) A similar experiment as performed under A was done with hydrophobins SC4 (also from S. commune) and ABH3 (from Agaricus bisporus). For both hydrophobins, similar results were obtained as for SC3, indicating that a low pH treatment at room temperature can be used for the stabilization of many types of hydrophobins.

D) A similar experiment as performed under A was done at different pH values, all in the presence of phosphate buffer. The results indicated that at low pH values 1 to 4, the rate of the β-sheet transition is faster than at higher pH values. Even at pH 7, the transition could be achieved at room temperature but it takes at least an overnight incubation. Thus, the lower the pH, the faster assembly takes place.

E) In a similar experiment as performed under A, the ingredients were mixed in a different order. The results showed that there is no influence of the order of mixing on the end result, namely the β-sheet formation.

F) In a similar experiment as performed under A, CD spectra were scanned rapidly in a time course. The results show that it takes only minutes to reach the β-sheet end state at pH 2. It is clear from the spectra that the α-helical state is a transient intermediate state as was observed for self-assembly on the air-water interface (de Vocht et al., 2002).

G) In a similar experiment as performed under A, the samples were mixed with 3 μM Thioflavin T (a fluorescent probe for amyloid proteins that fluoresces only when bound to the assembled state of SC3 (Butko et al., 2001, Spectroscopic evidence for amyloid-like interfacial self-assembly of hydrophobin SC3, Biochem. Biophys. Res. Commun. 280:212-215; Wösten and de Vocht 2000, Hydrophobins, the fungal coat unraveled, Biochim. Biophys. Acta 1469:79-86; McKay et al. 2001, the hydrophobin EAS is largely unstructured in solutions and functions by forming amyloid-like structures, Structure 9:83-91). The samples were diluted in water in a 3 ml quartz cuvette and placed in a SPF-500C spectrofluorometer (SLM Aminco). A dramatic increase in fluorescence was observed for those samples that are in a 3-sheet state as was observed with CD. Thus, real amyloid structures were induced by low pH at room temperature.

H) Correlation between pH and temperature on β-sheet formation of SC3. In a similar experiment as performed under A, the correlation between pH and temperature on the β-sheet formation of SC3 was investigated.

Before the measurement, the SC3 was dissolved in an HCl/KCl buffer pH 2 or phosphate buffer pH 7 and CD spectra were measured after addition of PTFE at temperatures of 15., 25, 40 and 80° C. (α-helical). After the measurement, 4 μl of 10% TWEEN 20 was added (0.1% final concentration) and the effect on the β-sheet formation was observed.

TABLE 1
β-sheet formation after addition of TWEEN
20 at various pH values and temperatures.

| | pH 2 | pH 7 |
|---|---|---|
| α-helical | yes | yes |
| β sheet at 15° C. | After 30 minutes | — |
| β sheet at 25° C. | After 0 minutes | Overnight |
| β sheet at 40° C. | After 0 minutes | After 30 minutes |
| β sheet at 80° C. | After 0 minutes | After 0 minutes |

These data illustrate that pH and temperature have a large influence on the rate of β-sheet formation of hydrophobin on PTFE. At pH 2, β-sheet formation occurs immediately at temperatures above 15° C. At pH 7, higher temperatures result in a faster formation of β-sheet.

### Example 4

### Coatings

The CD experiments that are performed in Example 1 are typically with 10% coverage of the surface of the colloidal PTFE and are characterized by CD. In this Example, coatings of PTFE with hydrophobin are 100% covered (i.e., an excess of hydrophobin was used) and are characterized by water contact angles.

PTFE sheets of 1 cm² were thoroughly cleaned with 100% EtOH, washed with water and dried. Hydrophobin layers were obtained by incubating the PTFE sheets in 2 ml containers containing 2 ml of 100 μg/ml hydrophobin solution with optional additives. Incubations are typically done for 16 hours at 25° C. The sheets are washed three times with 10 ml of milliQ water and were left to dry. A subsequent detergent treatment to stabilize the hydrophobin layers was performed in fresh 2 ml container with 2% SDS, 0.1% TWEEN 20 or 1% PVOH at a specified pH and temperature. The sheets were washed again three times with 10 ml of milliQ water and left to dry. The hydrophilicity of the surface was determined with a Drop Shape Analysis System DSA 10 Mk2 apparatus (Krüss) by measuring the contact angle of 1 to 2 μl water with the surface (Table 2).

TABLE 2
Contact angles of various coatings on PTFE sheets.

| | Contact |
|---|---|
| Sample | angle |
| Bare PTFE | 120° |
| PTFE + SC3 (16 hours); 2% SDS treatment (boiling 10 | 40° |
| minutes) | |
| PTFE + SC3/1% PVOH (pH 2, 25° C., 16 hours) | 50° |
| PTFE + 1% PVOH (pH 2, 25° C., 16 hours) | 90° |
| PTFE + SC3/0.1% TWEEN 20 (pH 2, 25° C., 16 hours) | 70° |
| PTFE + 0.1% TWEEN 20 (pH 2, 25° C., 16 hours) | 120° |
| PTFE + SC3 (16 hours); 1% PVOH (pH 2, 25° C., 1 hour) | 40° |
| PTFE + SC3 (16 hours); 0.1% TWEEN 20 (pH 2, 25° C., | 40° |
| 1 hour) | |

The results clearly indicate that low contact angles are achieved at 25° C. The best results are obtained by the traditional hot SDS treatment (which is applied on an existing coating) and by a low pH treatment at room temperature on preformed coatings. Low contact angles could also be achieved by coating the PTFE sheets directly in a solution of hydrophobin and detergent at room temperature.

### Example 5

### Assay

A microtiterplate assay was developed for determining the concentration and functionality of a hydrophobin-containing solution. The assay can be used to screen for the best hydrophobin-producing strain or for selecting functional hydrophobins and mutants thereof. In a well, a total volume of 200 μl was prepared containing 0-150 μg/ml SC3, 0.4% colloidal PTFE (w/v), 3 μM Thioflavin T, 0.1% PVOH and 10 mM HCl/KCl (pH 2). The plate was incubated at 25° C. and controls were taken that each missed one of the components. The fluorescence was read with a fluorometer at different time intervals. The fluorescence was plotted against the SC3 concentration and the data points could be fitted with a straight line (FIG. 3).

### Example 6

### Solubilization and Stabilization of a Hydrophobin-Containing Solution Using PFA

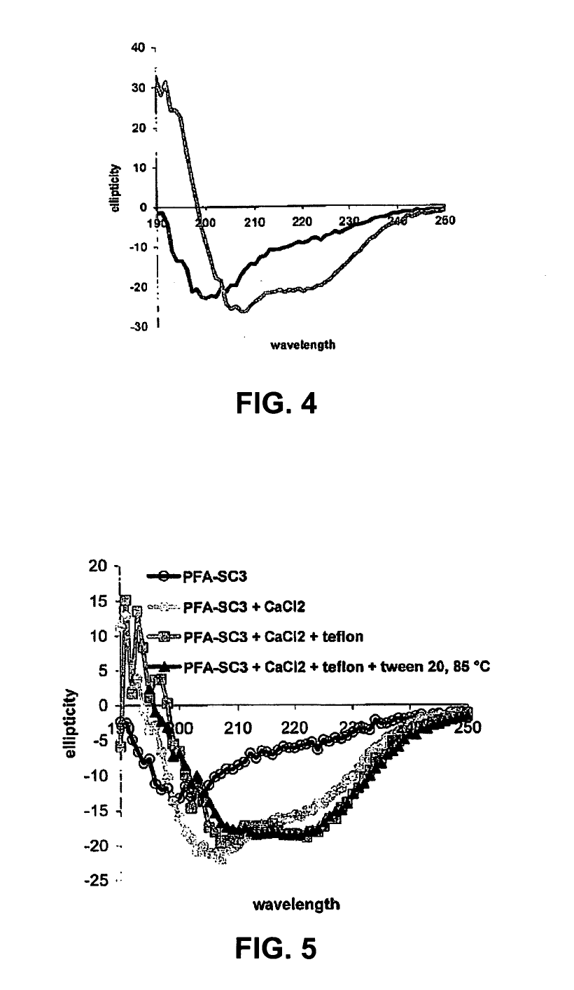

1 mg of purified freeze-dried SC3 (assembled or soluble) was dissolved in 1 ml of ice-cold performic acid (PFA) solution and kept on ice for 16 hours. A performic acid solution was made by mixing one part of 30% hydrogen peroxide with nine parts of concentrated formic acid, which was kept for one hour on table for activation and was pre-cooled for one hour on ice. The PFA solution, containing SC3, was applied to a PD-10 column (Pharmacia) equilibrated with milliQ water. The PFA-SC3 was collected in the first 3.5 ml of the column eluent in pure water. Fractions with lowered pH due to formic acid elution from the column were discarded. A solution with PFA-SC3 could be kept for months at 8° C. without changing its properties. CD experiments were performed with PFA-SC3 as described above, the results of which are shown in FIG. 4.

### Example 7

### Maldi-TOF Analysis of PFA-Treated Hydrophobins

Matrix-assisted laser desorption/ionization-time of flight mass spectrometry (MALDI-TOF MS) is a relatively novel technique in which a co-precipitate of a UV-light-absorbing matrix and a biomolecule is irradiated by a nanosecond laser pulse. Most of the laser energy is absorbed by the matrix, which prevents unwanted fragmentation of the biomolecule. The ionized biomolecules are accelerated in an electric field and enter the flight tube. During the flight in this tube, different molecules are separated according to their mass-to-charge ratio and reach the detector at different times. In this way, each molecule yields a distinct signal. The method is typically used for detection and characterization of biomolecules, such as proteins, peptides, oligosaccharides and oligonucleotides, with molecular masses between 400 and 350,000 Da. Maldi-TOF analysis was performed with PFA-SC3 and sinnapinic acid as matrix. This analysis revealed a mass increase of 600-700 Da of PFA-SC3 compared with SC3. For fully oxidized cysteine residues, a mass increase of 384 Da is expected when SC3 is oxidized with PFA. The extra mass is probably caused by Na/K salt adducts, resulting in a broad mass peak. Thus, the expected modification of hydrophobin by PFA could be confirmed by Maldi-TOF analysis.

### Example 8

### Heating PFA-SC3

Heating the PFA-SC3 and PTFE solution to 85° C. and adding 4 μl 10% TWEEN 20 resulted in partly dissolved protein and partly attached PFA-SC3 to PTFE.

### Example 9

### Obtain Stable PFA-SC3 Coating Triggered by Calcium

Calcium was added to neutralize the negative charge that is introduced by oxidation with performic acid and, therefore, to fancy coating into β-sheet conformation. The pH was lowered to below 1 by adding sufficient amounts of 10% TFA solution. Addition of 10 mM CaCl₂ to the PFA-SC3 solution resulted in a spectral change of unstructured (FIG. 5; open circles) towards a more α-helical structure (filled circles). Addition of PTFE resulted in the shift of the spectrum to a more β-sheet-like state (filled squares). A subsequent hot detergent treatment did not change anything to the spectrum, indicating that the end-state, namely β-sheet, has already been reached (filled triangles).

Thioflavin T was used to establish whether the β-sheet state (filled triangles) was indeed a β-sheet structure as with SC3 that was not treated with PFA. The experiment was performed as in Example 3G. Thioflavin T fluorescence increased dramatically with β-sheet PFA-SC3, indicating that, indeed, amyloid-like structures are formed.

### Example 10

### Effect of Concentration and Time on Coatings

PTFE sheets of 1.6 cm² were thoroughly cleaned with 100% EtOH, washed with water and dried. Hydrophobin layers were obtained by incubating the PTFE sheets in 2 ml containers containing 1.5 ml of hydrophobin solutions at a concentration of 300, 100, 50 or 5 μg/ml in a 50 mM sodium phosphate buffer pH7. Incubations were performed for 16 hours, 3 hours or 15 minutes at 25° C. The sheets were washed with milliQ water followed by different treatments. Half of the samples were incubated in 1% SDS at 100° C. for ten minutes and were washed with milliQ water. This hot detergent treatment was performed as positive control for stable coatings. Half of the samples that were not treated with hot SDS and half of the samples that were treated with hot SDS were incubated in 1.5 ml of 0.1% TWEEN 20 pH 7 for 15 minutes on a rotary table (20 rpm). The samples were washed with milliQ water. This TWEEN treatment was performed to determine the stability of the coating obtained under the various test conditions. All samples were left to dry. The hydrophilicity of the surface was determined with a Drop Shape Analysis System DSA 10 Mk2 apparatus (Krüss) by measuring the contact angle of 1 to 2 μl water with the surface (FIGS. 6A and 6B). The contact angle of water on untreated PTFE sheets is approximately 110°.

These results (see FIG. 6A) indicate that the concentration of the hydrophobin solution and the time of incubation both affect the obtained contact angles and the stability of the coatings (resistance towards washing in 0.1% TWEEN 20 pH 7). For example, coating at 300 μg/ml for 16 hours results in a stable coating with a low contact angle. In contrast, coating at 5 μg/ml for 16 hours results in an unstable coating with high contact angles and coating at 5 μg/ml for 15 minutes results in very high contact angles (FIG. 6A). As expected, the coatings treated with hot SDS were stable at all tested concentrations and incubation times (FIG. 6B).

### Example 11

### Effect of pH on Coatings

This example illustrates the contribution of a low pH and an increased hydrophobin concentration on the formation of a stable coating.

PTFE sheets of 1.6 cm were thoroughly cleaned with 100% EtOH, washed with water and dried. Hydrophobin layers were obtained by incubating the PTFE sheets in 2 ml containers containing 1.5 ml of hydrophobin solutions at a concentration of 300, 100 or 50 μg/ml. Hydrophobin solutions were made in either milliQ water pH 7 or milliQ water pH 4 (an increase to pH 7 was obtained by adding NaOH). Incubations were performed for 16 hours or 3 hours at 25° C. The sheets were washed with milliQ water followed by different treatments. Half of the samples were incubated in 1% SDS at 100° C. for ten minutes and were washed with milliQ water. Half of the samples that were not treated with hot SDS and half of the samples that were treated with hot SDS were incubated in 1.5 ml of 0.1% TWEEN 20 pH 7 for 15 minutes on a rotary table (20 rpm). The samples were washed with milliQ water. All samples were left to dry. The hydrophilicity of the surface was determined with a Drop Shape Analysis System DSA 10 Mk2 apparatus (Krüss) by measuring the contact angle of 1 to 2 μl water with the surface.

The results indicate that lowering the pH of the hydrophobin solution results in a decrease of the contact angle at concentrations below 300 μg/ml when coatings are incubated for 16 hours. Lowering the pH of the hydrophobin solution results in a decrease of the contact angle at all concentrations tested when shorter incubation is performed (three hours). In addition, lowering the pH results in a stable coating when coating is performed at 300 μg/ml for 16 hours (FIG. 7). The samples treated with hot SDS were stable at all tested concentrations and incubation times (not shown).

### Example 12

### Effect of Buffer on Coatings

PTFE sheets of 1.6 cm² were thoroughly cleaned with 100% EtOH, washed with water and dried. Hydrophobin layers were obtained by incubating the PTFE sheets in 2 ml containers containing 1.5 ml of hydrophobin solutions at a concentration of 300, 100 or 50 μg/ml. Hydrophobin solutions were made in either milliQ water pH 7 or phosphate buffer pH 7. Incubations were performed for 16 hours or 3 hours at 25° C. The sheets were washed with milliQ water followed by different treatments. Half of the samples were incubated in 1% SDS at 100° C. for ten minutes and were washed with milliQ water. Half of the samples that were not treated with hot SDS and half of the samples that were treated with hot SDS were incubated in 1.5 ml of 0.1% TWEEN 20 pH 7 for 15 minutes on a rotary table (20 rpm). The samples were washed with milliQ water. All samples were left to dry. The hydrophilicity of the surface was determined with a Drop Shape Analysis System DSA 10 Mk2 apparatus (Krüss) by measuring the contact angle of 1 to 2 μl water with the surface.

The results (FIG. 8) show that the presence of buffer in the hydrophobin solutions results in a decrease of the contact angle at concentrations below 300 μg/ml when coatings are performed for 16 hours. The presence of buffer decreases contact angles at all concentrations tested when a short incubation (three hours) is performed. The presence of buffer did not affect the stability of the coatings. The samples treated with hot SDS were stable at all tested concentrations and incubation times (not shown).

What is claimed is:

1. A method for providing the surface of an object with a hydrophobin layer, the method comprising:
contacting at least a part of the object with a hydrophobin-containing solution to form a hydrophobin layer on the surface of the object, and
exposing the hydrophobin layer to a pH below 7.

2. The method according to claim 1, wherein exposing the hydrophobic layer to a pH below 7 comprises exposing the hydrophobin layer to a pH selected from the group of pHs consisting of below 4 and below 2.

3. The method according to claim 1, wherein exposing the hydrophobic layer to a pH below 7 comprises exposing the hydrophobin layer to a pH below 7 in the presence of a detergent.

4. The method according to claim 3, wherein the detergent is selected from the group consisting of TWEEN-20, TWEEN-80, polyvinyl alcohol, TRITON-X100, and sodium dodecylsulfate.

5. The method according to claim 3, wherein the detergent is present in concentration in an amount selected from the group consisting of at least 0.001% wt./vol, at least 0.01% wt./vol., and at least 1% wt./vol.

6. The method according to claim 1, wherein the hydrophobin-containing solution comprises a supernatant of a culture medium of an organism that secretes a hydrophobin.

7. The method according to claim 6, further comprising:
culturing the organism, and
adding a detergent to the supernatant of the culture medium after culturing the organism.

8. The method according to claim 6, further comprising:
culturing the organism in the presence of a detergent.

9. The method according to claim 6, wherein the organism produces a detergent-like compound.

10. The method according to claim 1, wherein contacting at least a part of an object with a hydrophobin-containing solution is performed at a temperature selected from the group of temperatures consisting of below about 30° C., below about 25° C., and below about 15° C.

11. The method according to claim 1, wherein the hydrophobin solution is present in an amount selected from the group consisting of at least 5 mg hydrophobin per liter, at least 10 mg hydrophobin per liter, and at least 20 mg hydrophobin per liter.

12. The method according to claim 1, wherein the object is selected from the group consisting of a window, a contact lens, a biosensor, a medical device, a container for performing an assay or storage, the hull of a vessel, a frame or bodywork of a car, a solid particle, a porous material, and a textile.

13. A method for providing the surface of an object with a layer of a hydrophobin, the method comprising:
contacting an object with a hydrophobin-containing solution, wherein the hydrophobin-containing solution comprises hydrophobin that has been treated with performic acid.

14. The method according to claim 13, wherein the pH of the hydrophobin-containing solution is selected from the group consisting of below 7, below 4, and below 1.

15. The method according to claim 13, wherein the hydrophobin-containing solution further comprises divalent metal ions.

16. The method according to claim 13, wherein the hydrophobin-containing solution further comprises Ca²⁺ ions.

17. The method according to claim 13, wherein the object is selected from the group consisting of a window, a contact lens, a biosensor, a medical device, a container for performing an assay or storage, the hull of a vessel, a frame or bodywork of a car, a solid particle, a porous material, and a textile.

18. A method for optimizing the conditions for providing the surface of an object with a hydrophobin layer by contacting at least part of the surface with a hydrophobin-containing solution at room temperature, the method comprising:
determining the effect of at least two parameters on the formation of a hydrophobin layer in the surface of the object wherein the parameters are selected from the group consisting of pH, incubation time, concentration of hydrophobin in the hydrophobin-containing solution and presence of a buffer in the solution.

19. A culture medium for culturing a hydrophobin-producing organism, comprising a detergent.

20. The culture medium of claim 19, together with a hydrophobin-producing organism.

21. A method for providing an object with a hydrophobic layer comprising:
using a performic acid-treated hydrophobin to provide the object with the hydrophobic layer.

22. A method for preventing assembly of hydrophobin in a hydrophobin-containing solution, said method comprising:
including a detergent in the hydrophobin-containing solution
so as to prevent assembly of hydrophobin in the hydrophobin-containing solution.
